(19) United States
(12) Patent Application Publication
(10) Pub. No.: US 2021/0199643 A1
(43) Pub. Date: Jul. 1, 2021

(54) FLUID CLASSIFICATION

(71) Applicant: Hewlett-Packard Development Company, L.P., Spring, TX (US)

(72) Inventors: Sunil Bharitkar, Palo Alto, CA (US); Caitlin DeJong, Palo Alto, CA (US); Anita Rogacs, San Diego, CA (US); Steven J. Simske, Ft. Collins, CO (US)

(73) Assignee: Hewlett-Packard Development Company, L.P., Spring, TX (US)

(21) Appl. No.: 16/761,829

(22) PCT Filed: Jan. 16, 2018
(86) PCT No.: PCT/US2018/013817

§ 371 Date: May 6, 2020

(51) Int. Cl. G01N 33/497; G01N 33/00; G01N 33/52; G06N 3/04

(52) U.S. Cl. CPC G01N 33/497; G01N 33/0031; G06N 3/04; G01N 33/0063; G01N 33/52

(57) ABSTRACT

Fluid classification may include: receiving sensed data for the fluid; modeling the sensed data in a frequency domain; synthesizing a model of the sensed data from the frequency domain to a time domain response and converting the time domain response to a time frequency graphical representation in the form of a color map. Predetermined characteristics of the time frequency graphical representation are identified through computer vision and compared to at least one corresponding signature characteristic of a predetermined fluid type to identify the fluid as a fluid type.

### BACKGROUND

The identification and classification of unknown substances or samples, sometimes referred to as fluids, may be utilized in a variety of different fields for a variety of different purposes. For example, gaseous fluids may be analyzed and classified to indicate air quality. Tissue or blood sample fluids may be analyzed and classified to indicate the health of the host from which the tissue or blood sample was taken.

### BRIEF DESCRIPTION OF THE DRAWINGS

FIG. 1 is a schematic diagram of an example fluid classification system.

FIG. 2 is a flow diagram of an example method for populating an example fluid classification library or model.

FIG. 3 is a flow diagram of an example method for classifying an unknown fluid.

FIG. 4 is a flow diagram illustrating examples of the classifying of the unknown fluid.

FIG. 5 is a flow diagram of an example method for populating an example fluid classification model and classifying an unknown fluid using the model.

FIG. 6A is a diagram of an example model of cancerous SERS spectra training data model in a frequency domain.

FIG. 6B is a diagram of a time domain response of the cancerous SERS training data synthesized from the model of FIG. 6A.

FIG. 7A is a diagram of an example model of healthy SERS spectra training data model in a frequency domain.

FIG. 7B is a diagram of a time domain response of the healthy SERS training data synthesized from the model of FIG. 7A.

FIG. 8 is a diagram of an example Hamming window used to form a time frequency representation, in the form of a spectrogram, of a time domain response.

FIG. 9 is a diagram illustrating an example time frequency representation in the form of a spectrogram resulting from application of a windowed short time Fourier transform with overlapping windows to the time domain response.

FIG. 10 is a diagram of an example display illustrating the concurrent presentation of color maps generated from various samples/fluids.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed herein are example fluid classification systems, methods and databases that facilitate the identification or classification of fluids being tested. The fluids being tested may in a liquid or gas phase. The fluids being tested may include a single analyte or multiple analytes. Such testing may identify a single analyte or a group of analytes of the fluid.

The systems, methods and databases convert sensed data into color maps which are then optically analyzed by computer vision to identify or classify the fluid (or it's analyte(s)) of interest. The systems, methods and databases may output color maps or graphics that provide a visibly detectable correlation between a fluid being tested and a predetermined fluid such that a person may visibly appreciate the basis for the fluid classification and identification.

Disclosed herein are example classification systems, methods and database that facilitate the use of SERS sensors and data analytics to distinguish between media exposed to different cell types. The data may be represented as a spectral response comprising the “signature” of the fluids at different wavelengths. The spectral data obtained from the sensors are transferred to a time frequency distribution by synthesizing a time domain approximation to the spectral data and then performing a time frequency representation. The representation is transformed to a color map to train a stack of convolutional neural network (CNN) and fully connected feedforward neural network for classification.

Disclosed herein is an example fluid classification method that may include: receiving sensed data for the fluid; modeling the sensed data in a frequency domain; synthesizing a time-domain model of the sensed data from the frequency domain to a time domain response, using inverse Fourier transform, and converting the time domain response to a time frequency graphical representation which then forms the basis for a color map. Predetermined characteristics of the time frequency graphical representation are identified through computer vision and compared to at least one corresponding signature characteristic of a predetermined fluid type to identify the fluid as a fluid type.

Disclosed herein is an example non-transitory computer-readable medium that contains instructions to direct a processing unit to perform fluid classification. The classification of the fluid is performed by receiving sensed data for the fluid, modeling the sensed data in a frequency domain, synthesizing a model of the sensed data from the frequency domain to a time domain response and converting the time domain response to a time frequency graphical representation in the form of a color map. The instructions further direct the processing unit to identify a predetermined characteristics of the time frequency graphical representation through computer vision and classifying the fluid by comparing identified characteristics of the graphical representation to at least one corresponding signature characteristic of a predetermined fluid type.

Disclosed herein is an example database for fluid classification. The database may include fluid classifications. Each fluid classification may comprise predetermined visual characteristics of the fluid classification corresponding to application of a convoluted neural network to a time-frequency representation of spectrographic data for the fluid classification.

In one implementation, the database of fluid classifications is formed by receiving second sensed data for the predetermined fluid type, modeling the second sensed data in a frequency domain, synthesizing a model of the second sensed data from the frequency domain to a time domain response, and converting the time domain response for the second sensed data to a second time-frequency graphical representation in the form of a second color map. Predetermined characteristics of the second time-frequency graphical representation are identified through computer vision. An association of the second identified characteristics of the second graphical representation to at least one signature characteristic of the predetermined fluid type is stored to form the database.

FIG. 1 schematically illustrates an example fluid classification system 20. Classification system 20 converts sensed data into color maps which are then optically analyzed by computer vision to identify or classify the fluid of interest. In one implementation, classification system 20 may be used to build a fluid classification library of different classified fluids and their associated signature characteristics. In one implementation, classification system 20 may utilize a fluid classification library to identify and classify unknown substances or fluids. Fluid classification system 20 comprises sensed data input 22, indicator 24, processing unit 26, fluid classification library 28 and non-transitory computer-readable medium or memory 30.

Sensed data input 22 comprise an electronic input or electronic hardware by which sensed data is transmitted to processing unit 26. In one implementation, sensed data input 22 receives raw signals from at least one sensor, wherein processing unit 26 processes the raw signals for further use. In another implementation, sensed data input 22 comprises electronic input by which processed data, based upon the sensed data, is received and transmitted a processing unit 26.

In one implementation, sensed data input 22 receives data from an optical sensor. In one implementation, data input 22 receives spectrographic comprising Raman spectroscopy data are luminescence data. In one implementation, since data input 22 receives data from at least one impedance sensor. In yet other implementations, data input 22 receives other forms of data from other types of substance sensors or detectors.

Indicator 24 comprises hardware or electronics by which an identification of a fluid or its classification is output. In one implementation indicator 24 may comprise an optical or audible indicator. In one implementation, indicator 24 may comprise at least one light emitting diode which is illuminated or which is illuminated with different colors based upon the determined classification for a previously unknown fluid. In one implementation, indicator 24 may comprise a display, such as a touchscreen or monitor. For example, in some implementations, indicator 24 may comprise a display which concurrently presents a generated color map for the unknown fluid of interest and at least one color map for already identified substances or predetermined fluids such that a person may visibly discern the similarities and differences between color maps and appreciate any basis for conclusions made regarding the identification or classification of the unknown fluid. In one implementation, the display may present the generated color map for the unknown fluid and the at least one color map for the already known fluids are substances in a side-by-side manner. In some implementations, the color maps may be partially overlap on the display two more directly correlate different characteristics of the tomb color maps that are similar or distinct from one another in which serve as a basis for the classification. In such implementations, those correlating characteristics of the color maps which serve as a basis for the classification decision are identified or indicated on the display.

Processing unit 26 comprises electronics or hardware that carries out or follow the instructions provided in medium 30 to classify a previously unknown fluid (sample) based upon data received through sensed data input 22 and signature characteristic stored in fluid classification library 28. In some implementations, processing unit 26 also follows instructions provided medium 32 build or supplement the fluid classification library 28 with signature characteristics of previously identified substances or fluids. Processing unit 26 may comprise a single processing unit or may be distributed amongst multiple processing units to carry out different operations with respect to the data received through input 22.

Fluid classification library 28 comprise a database for fluid classification. Fluid classification library 28 comprises various fluid classifications. Each fluid classification comprises at least one predetermined visual characteristic of a fluid classification output or resulting from the application of computer vision to a generated color map for a fluid that was previously identified through other techniques (potentially more time-consuming and costly). In one implementation, a convoluted neural network is applied to the color map to identify signature characteristics of the color map. In one implementation, the convoluted neural network is applied to a time-frequency representation of spectrographic data for the previous identified substance or fluid.

In one implementation, fluid classification library 28 comprises a computer-readable lookup table comprising individual entries for different fluid types or different fluid classifications. Each fluid type or classification entry has associated values or ranges of values for various characteristics or attributes of the color map (values for selected portions of the color map) that is associated with the particular fluid type or classification. In one implementation, each fluid type or classification entry has associated values or ranges of values from the time-frequency distribution (e.g., spectrogram) such as decay time (n_{d}) as derived relative to the peak intensity at a given frequency (e.g., |(n_{d},wᵢ)=20 log₁₀|Snd(e^{jw}ᵢ)|˜−20 dB).

Non-transitory computer-readable medium 30 comprises software and/are integrated circuitry that include provide instructions to processing unit 26 for adding entries to fluid classification library 28 and/or classifying a fluid of interest. Medium 30 comprises various modules sets of instructions are carrying out different processes in the classification of a fluid. Medium 30 comprises frequency domain modeler 40, time domain response to the size or 42, color map generator 44, computer vision analyzer 46 and fluid identifier 48.

Frequency domain modeler comprises circuitry or programming/code embodying instructions that direct processing unit 26 to model the sensed data received through input 22 in a frequency domain. Such modeling may involve baseline correction of the sample signals from the sensor.

Time domain response synthesizer 42 directs processor 26 to synthesize the frequency domain modeler produced by modeler 40 from the frequency domain to a time domain response. In one implementation, the time domain response is generated using a finite-impulse-response (FIR) sampling approach which linearly interpolates a desired frequency response onto a dense grid and then uses an inverse Fourier transform along with an N-point Hamming window to obtain an N-duration time-domain response. In one implementation, a value of 8192 for N was utilized respect to surface enhanced Raman spectroscopy (SERS) spectra data.

Color map generator 44 comprise circuitry or programming/code embodying instructions that direct processor 26 to convert the time domain response to a time frequency graphical representation which forms the basis of a color map. In one implementation, color map generator 44 outputs a time-frequency representation in the form of a spectrogram. In one implementation, the spectrogram is produced using a windowed short time Fourier transform with overlap between the windows. For example, in one implementation, for a signal s(m) with the windowing the function w(m), the short time Fourier transform STFT Sₙ(e^{jwi}) at time n and frequency wᵢ is expressed as follows:

$${S}_{n} ( {e}^{j ω i} ) = {\sum }_{m} s ( m ) ω ( n - m ) {e}^{- j ω im}$$

The time frequency representation by the spectrogram facilitates capture of the temporal-spectral behavior of time-domain signal used to approximate the data, such as SERS spectra. The conversion of the time domain response to a time frequency graphical representation in the form of a spectrogram using short time Fourier transform results in those components of the FIR time-domain signal that fit narrow peaks ringing longer whereas components that fit wider peaks ring” less in duration. After resampling the data in the frequency-domain and inverse Fourier transforming the result, a time-domain signal is synthesized which is then finally transformed to the time-frequency representation. The time-frequency representation is then converted into a color map. For example, time frequency domain may be transformed to red/green/blue (R/G/B) or grayscale channels representing an image of the spectrogram. In other implementations, the time domain response may be converted to other time-frequency representations. For example, in some implementations, in lieu of being converted to a spectrogram, the time-domain response may be converted to a Wigner-Ville time frequency representation which is then utilized as a basis for generating the color map.

Computer vision analyzer 46 comprises circuitry or programming/code embodying instructions that direct processing unit to analyze the color map and identify predetermined optical characteristics of the time frequency graphical representation/color map through computer vision. In one implementation, computer vision analyzer 46 comprise a cascade of CNN and a fully connected feedforward artificial neural network for identifying predetermined characteristics of the time frequency graphical representation. In other implementations, computer vision analyzer 46 may comprise instructions to direct processor 26 to utilize other computer vision techniques, such as Support Vector Machines (SVM), Bayesian classifier, Forest regression to identify predetermined characteristics of the time graphical representation, the color map.

In implementations where system 20 is building or supplementing fluid classification library 28, computer vision analyzer 46 may store values for the predetermined characteristics along with the associated (previously identified) substance or fluid in fluid classification library 28. In implementations where the values for the predetermined characteristics are for a substance or fluid that is yet to be classified are identified, the values are transmitted to fluid identifier 48.

Fluid identifier 48 comprises circuitry or programming/code embodying instructions to direct processing unit 26 to classify/identify the unknown substance or fluid by comparing the identified values for the predetermined characteristics of the graphical representation to at least one corresponding signature value or range of values for a particular fluid classification or type as obtained from fluid classification library 28. For example, the values for a particular characteristic obtained from the color map from the unknown fluid may be compared to the values for the same particular characteristic obtained from the color map from a previously identified substance or fluid stored in library 28. Based upon this comparison, the unknown fluid may be classified.

In one implementation, the classification of the unknown fluid may be based upon similarities between the values for the predetermined characteristics of the color maps for the unknown fluid and the previously identified fluid (library or database entry). If a sufficient similarity exists, the unknown fluid may be classified as being in the same class or of the same type as the previously identified fluid. For example, values for an unknown tissue sample or blood sample may be compared to corresponding values for tissue or blood sample previous identified as being cancerous, wherein the unknown tissue sample or blood sample may likewise be classified as cancerous if sufficient similarities are identified between the values obtained from the color maps for the blood sample/tissue sample and the previously identified cancerous blood sample/tissue sample.

In another implementation, the classification of the unknown fluid may be based upon differences between the values for the predetermined characteristics of the color maps for the unknown fluid and the previous identified fluid. For example, values for an unknown tissue sample or blood sample may be compared to corresponding values for a tissue or blood sample previous identified as being cancerous, wherein the unknown tissue sample or blood sample may be classified as healthy if sufficient dissimilarities are identified between values obtained from the color maps for the blood sample/tissue sample and the previously identified cancerous blood sample/tissue sample.

In one implementation, fluid identifier 48 further direct processing unit 26 to concurrently display color map for the unknown fluid and the color map for the previously identified fluid. In one implementation, fluid identifier 48 direct processing unit 26 to display the color maps in a side-by-side fashion. In another implementation, fluid identifier 48 direct processing unit 26 to at least partially overlap the color maps. In some implementations, fluid identifier 48 additionally direct processing unit 28 to indicate those peaks, amplitudes or other predetermined characteristics that were utilized to classify the unknown fluid. The indication may be by way of color, annotations, markings or the like. Such a fashion, the person viewing the display may visibly appreciate the similarities and/or differences visibly represented by the color map and resulting in the particular classification of the unknown fluid.

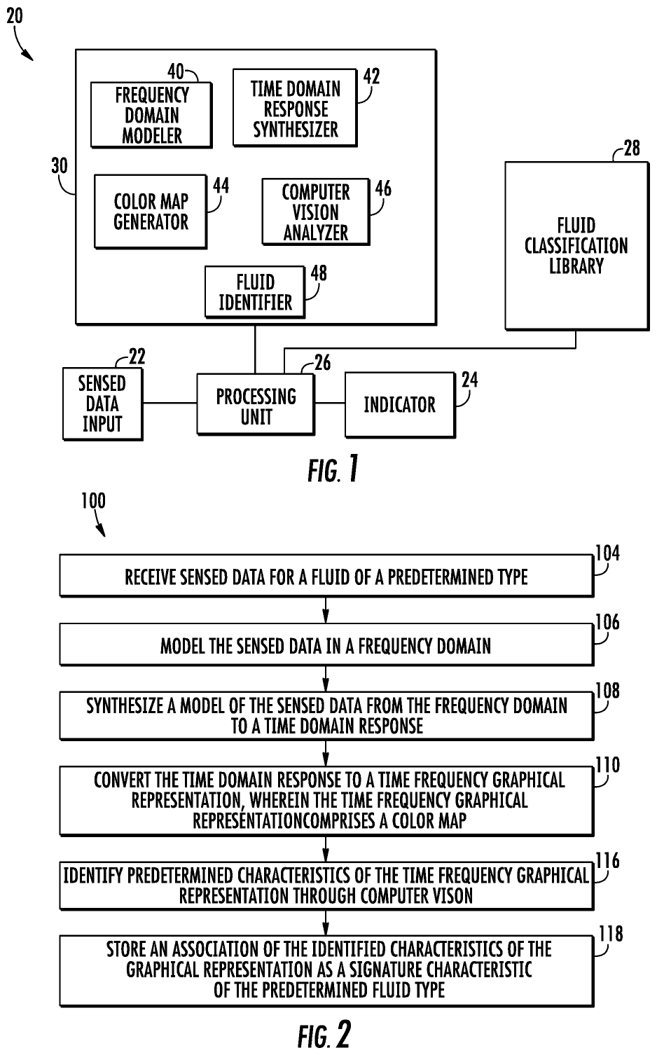

FIG. 2 is a flow diagram of an example method 100 for producing a fluid classification database or library, such as library 28. Method 100 facilitates subsequent classification of unknown fluid through optical analysis of a color map generated from sensed data. Although method 100 is described in the context of being carried out by system 20, it should be appreciated that method 100 may be likewise carried out with other similar classification systems.

As indicated by block 104, processing unit 26 receives sensed data for a fluid of a predetermined type, a fluid for which an identity or classification has already been determined by other techniques. In one implementation, the sensed data may comprise spectroscopy data, such as surface enhanced Raman spectroscopy data, or fluorescence data. In another implementation, the sensed data may comprise impedance signal data or other forms of data resulting from interaction with the fluid of the predetermined type.

As indicated by block 106, processing unit 26, following instructions provided by a frequency domain modeler 40, models the sensed data in a frequency domain. As indicated by block 108, processing unit 26, following instructions provided by time domain response synthesizer, synthesizes a model of the sensed data from the frequency domain to a time domain response. In one implementation, the time domain response is performed using a finite-impulse-response (FIR) sampling approach which linearly interpolates a desired frequency response onto a dense grid and then uses an inverse Fourier transform along with an N-point Hamming window to obtain an N-duration time-domain response. In one implementation, a value of 8192 for N was utilized respect to surface enhanced Raman spectroscopy (SERS) spectra data.

As indicated by block 110, processor 26, following instructions provided by color map generator 44, converts the time domain response to a time frequency graphical representation the form of a color map. In one implementation, the time-frequency representation is in the form of a spectrogram. In one implementation, the spectrogram is produced using a windowed short time Fourier transform with overlap between the windows. For example, in one implementation, for a signal s(m) with the windowing the function w(m), the short time Fourier transform STFT Sₙ(e^{jwi}) at time n and frequency wᵢ is expressed as follows:

$${S}_{n} ( {e}^{j ω i} ) = {\sum }_{m} s ( m ) ω ( n - m ) {e}^{- j ω im}$$

The time frequency representation by the spectrogram facilitates capture of the temporal-spectral behavior of time-domain signal used to approximate the data, such as SERS spectra. The conversion of the time domain response to a time frequency graphical representation in the form of a spectrogram using short time Fourier transform results in those components of the FIR time-domain signal that fit narrow peaks ringing longer whereas components that fit wider peaks ring” less in duration. After approximating the data by frequency response was inverse is a time-domain signal that is then represented as the time frequency domain by the image, the time frequency domain is then converted into a color map. For example, time frequency domain may be transformed to red/green/blue channels representing an image of the spectrogram. In other implementations, the time domain response may be converted to other time-frequency representations. For example, in some implementations, in lieu of being converted to a spectrogram, the time-domain response may be converted to a Wigner-Ville time frequency representation which is then utilized as a basis for generating the color map.

As indicated by block 116, processor 26, following instructions provided by computer vision analyzer 46, analyzes the color map and identifies values for predetermined optical characteristics or optical parameters from the time frequency graphical representation/color map through computer vision. In one implementation, computer vision analyzer 46 comprise a cascade of CNN and a fully connected artificial neural network for identifying predetermined characteristics of the time frequency graphical representation. In other implementations, computer vision analyzer 46 may comprise instructions to direct processor 26 to utilize other computer vision techniques, such as Support Vector Machine (SVM), Bayes discriminator, etc. to identify predetermined characteristics of the time graphical representation, the color map.

As indicated by block 118, processor 26, operating in a fluid classification building or supplementing mode pursuant to instructions provided by fluid identifier 48, stores the determined or identified values for the predetermined characteristics/parameters of the color map along with the associated (previously identified) substance or fluid in fluid classification library 28. In one implementation, the identified values for the predetermined characteristics are used to establish new library or database entries for the previously identified substance or fluid. In another implementation, the identified values for the predetermined characteristics are used to establish a larger statistical base for the value or range of values for each of the predetermined characteristics or parameters that are used to identify an unknown fluid as being of the same classification or type as the previously identified substance or fluid.

FIG. 3 is a flow diagram of an example method 200 for classifying an unknown fluid (substance). Method 200 is similar to method 100 described above except that method 200 is carried out using sensed data from an unknown fluid and except that instead of storing the determined values for selected parameters or characteristics of the color map as part of the fluid classification library 28, method 200 comprises block 218.

In block 218 processing unit 26, following instructions provided by fluid identifier 48, classifies the unknown fluid by comparing the identified predetermined characteristics of the graphical representation or color map (their values) to at least one signature characteristic of a predetermined fluid type (its values or value range). Once the unknown fluid has been classified or its type has been identified, processing unit 26 outputs the classification or type using indicator 24.

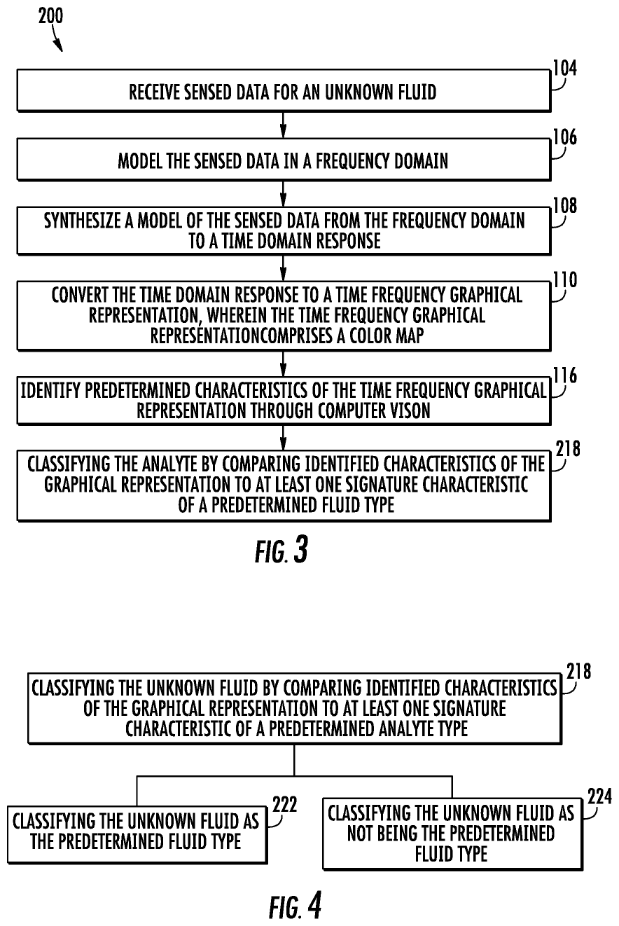

FIG. 4 is a flow diagram illustrating, in more detail, the classification of an unknown fluid pursuant to block 218. As indicated by block 222, in a first mode of operation, fluid identifier 48 may direct processing unit 26 to classify the unknown fluid as the predetermined fluid type, as being of the same type or classification as the predetermined fluid. Such a classification or determination may be based upon the values for the predetermined characteristics or parameters of the color map for the unknown fluid satisfying predetermined thresholds or falling within value ranges that correspond to the predetermined fluid. For example, in one implementation, and fluid classification may have a value of between A and B for a particular characteristic of the color map associated with the fluid classification X, i.e., the predetermined fluid type. In response to the unknown fluid having an associated color map with a value that is also between A and B for the same particular characteristic, processing unit 26 may classify the unknown fluid as X.

In one implementation, the fluid classification library 28 may include values for a set of parameters from different portions of a first color map generated from a tissue or blood sample pre-identified as being cancerous. To determine whether or not a tissue or blood sample taken from a subject being diagnosed is also cancerous, system 20 may generate a second color map based upon data sensed from the unclassified tissue or blood sample. Computer vision analyzer 46 may determine values for the same set of parameters from the same different portions of the second color map and compare the determined values for the second color map to the corresponding values of the first color map. In response to sufficient statistical similarity or in response to the satisfaction of a similarity threshold, processing unit 26, following instructions of fluid identifier 48, may classify the tissue or blood sample from the subject being diagnosed as cancerous.

As indicated by block 224, in a second mode of operation, fluid identifier 48 may direct processing unit 28 to classify a fluid as not being predetermined fluid type. Such a classification or determination may be based upon the values for the predetermined characteristics or parameters of the color map for the unknown fluid satisfying predetermined dissimilarity thresholds or falling outside of value ranges that correspond to the predetermined fluid. For example, in one implementation, and fluid classification may have a value of between A and B for a particular characteristic of the color map associated with the fluid classification X, i.e., the predetermined fluid type. In response to the unknown fluid having an associated color map with a value that is outside of A and B for the same particular characteristic, processing unit 26 may classify the unknown fluid as not X.

In one implementation, the fluid classification library 28 may include values for a set of parameters from different portions of a first color map generated from a tissue or blood sample pre-identified as being “healthy”. To determine whether or not a tissue or blood sample taken from a subject being diagnosed is also healthy, system 20 may generate a second color map based upon data sensed from the unclassified tissue or blood sample. Computer vision analyzer 46 may determine values for the same set of parameters from the same different portions of the second color map and compare the determined values for the second color map to the corresponding values of the first color map. In response to sufficient statistical dissimilarity, processing unit 26, following instructions of fluid identifier 48, may classify the tissue or blood sample from the subject being diagnosed as not “healthy”. As such point, additional testing or diagnosis may be called for to more specifically diagnose the type of element or cancer associated with the tissue or blood sample.

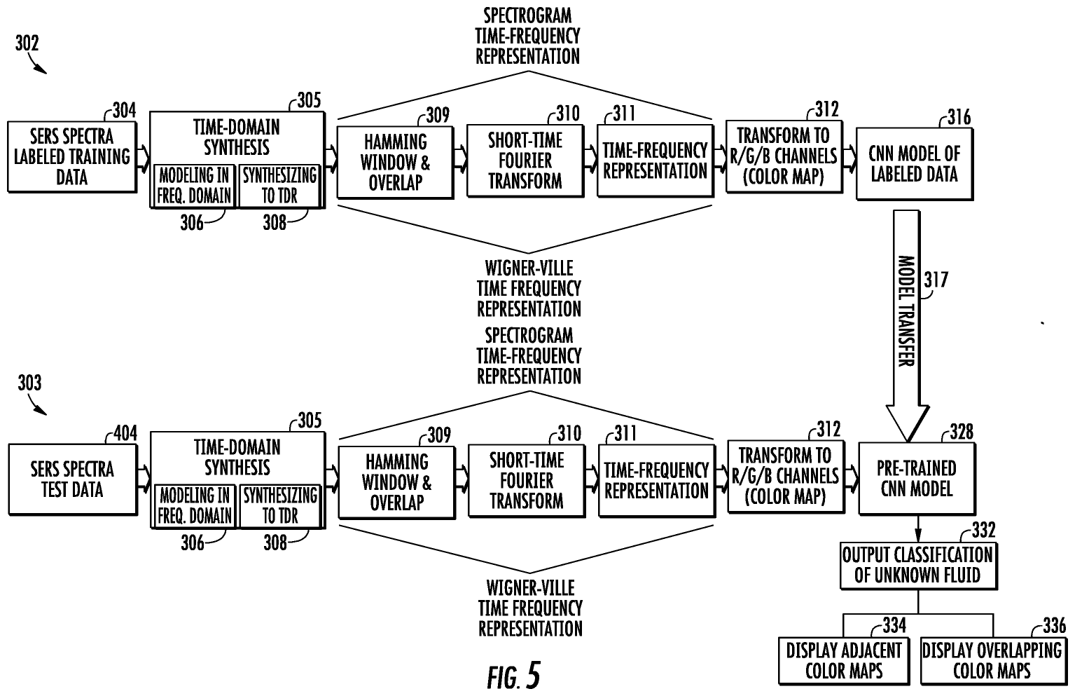

FIG. 5 is a flow diagram of an example method 300 for building or supplementing a fluid classification library and for classifying fluids of unknown classification. FIG. 5 illustrates two branches of method 300: a first branch 302 in which a system, such a system 20, is “trained”, producing a fluid classification library or model; and a second branch 303 which utilizes the fluid classification library model to classify a fluid of unknown type or classification. Each of branches 302, 303 utilizes same steps for generating a color map and extracting values from the generated color map. Although method 300 is described in the context of classifying a tissue or blood sample as being either healthy or cancerous using sensed SERS spectra data taken from the tissue or blood sample, it should be appreciative method 300 may likewise be utilized for classifying other types of fluid, whether gaseous or liquid in form, with respect to other classifications. Although method 300 described in the context of being carried out utilizing system 20 described above, it should be appreciated that method 300 may be carried out using other similar classification systems.

As indicated by block 304, processing unit 26 receives sensed “training” data for a fluid of a predetermined type, a fluid for which an identity or classification has already been determined by other techniques. In the example illustrated, the sensed data may comprise spectroscopy data, such as surface enhanced Raman spectroscopy data. In another implementation, the sensed data may comprise impedance signal data or other forms of data resulting from interaction with the fluid of the predetermined type.

In one implementation, different biofluids containing healthy cells and containing cancer cells are placed in respective mediums and are sensed using gold-based sensors are SERS substrate. The different cells (breast, cervical cancer as well healthy cervical epithelium) are cultured in mediums that bathe the cells to nourish the cells facilitating collection of cellular output. Surface enhanced Raman scattering signatures are derived using the SERS substrates, wherein the surface enhanced Raman scattering signatures serve as the training data received in block 304. Bio fluids for classification may be processed in a similar fashion to provide the SERS spectra test data received in block 404.

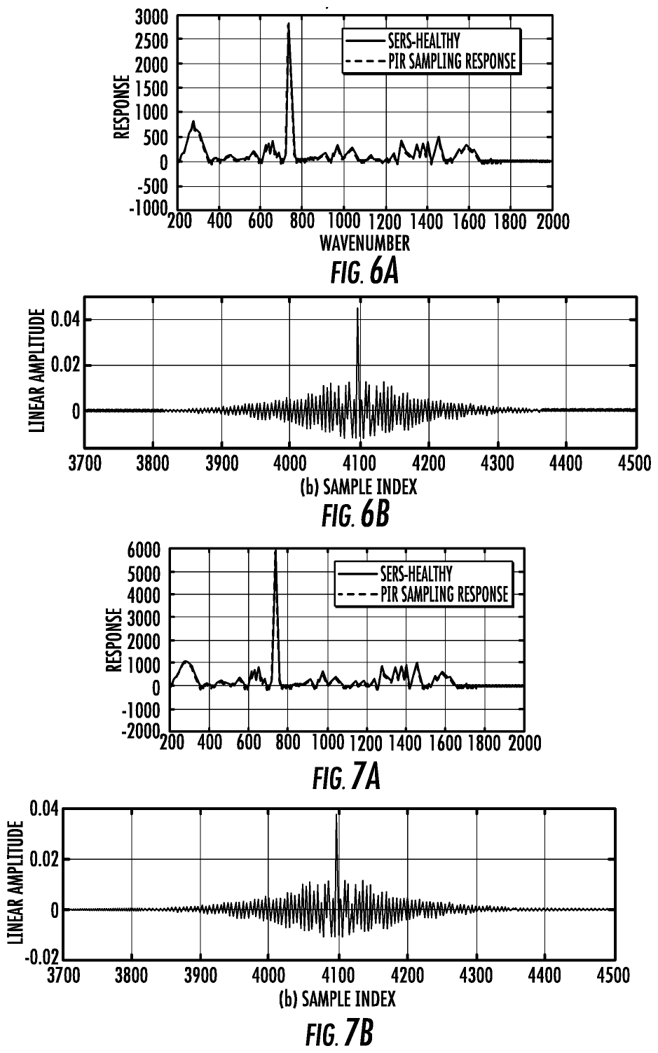

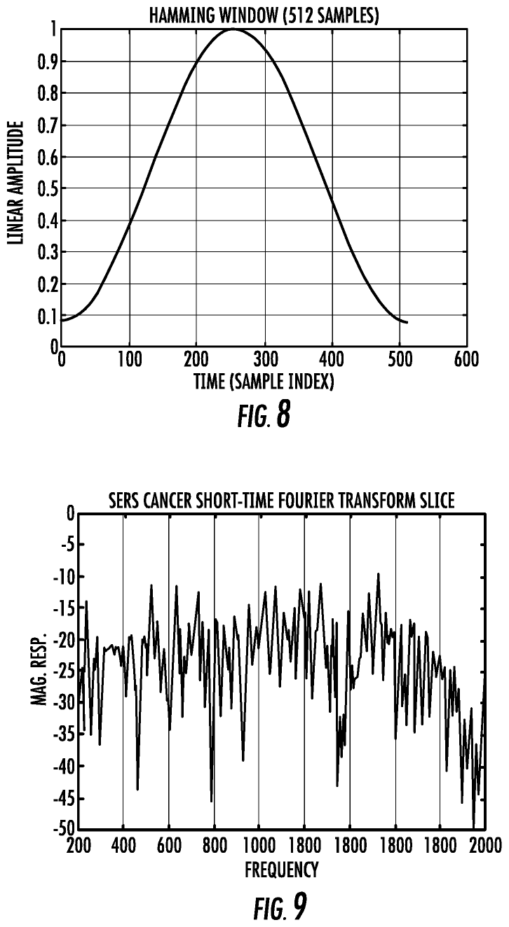

As indicated by block 305, processing unit 26, carrying out instructions provided by frequency domain modeler 40 and time domain response synthesizer 42 carry out time-domain synthesis. As indicated by block 306, processing unit 26, following instructions provided by a frequency domain modeler 40, models the sensed data in a frequency domain. FIGS. 6A and 7A illustrate examples of the modeling of SERS spectra data in the frequency domain for cancerous and healthy biological samples taken from first and second subjects, respectively.

As indicated by block 308, processing unit 26, following instructions provided by time domain response synthesizer, synthesizes a model of the sensed data from the frequency domain to a time domain response. In one implementation, the time domain response is performed using a finite-impulse-response (FIR) sampling approach which linearly interpolates a desired frequency response onto a dense grid and then uses an inverse Fourier transform along with an N-point Hamming window to obtain an N-duration time-domain response. In one implementation, a value of 8192 for N was utilized respect to surface enhanced Raman spectroscopy (SERS) spectra data. FIGS. 6B and 7B illustrate examples of the synthesis of the model shown in FIGS. 6A and 7A, respectively, into time domain responses. As shown by FIGS. 6B and 7B, the two samples or “fluids” exhibit different time domain responses. The temporal amplitudes and decay rates are different with respect to one another.

As indicated by blocks 309, 310 and 311, processor 26, following instructions provided by color map generator 44, converts the time domain response to a time frequency representation in the form of a spectrogram. In the example illustrated, the spectrogram is produced using a windowed short time Fourier transform with overlap between the windows. As indicated by block 309, a Hamming window is applied to a block of the time domain response. FIG. 8 illustrates one example Hamming window applied to a block of 512 data points (or samples).

As indicated by block 310, a short time Fourier transform is applied to the windowed time domain response with overlap between the windows to produce the time frequency representation indicated by block 311. In one implementation, for a signal s(m) with the windowing the function w(m), the short time Fourier transform STFT Sₙ(e^{jwi}) at time n and frequency wᵢ is expressed as follows:

$${S}_{n} ( {e}^{j ω i} ) = {\sum }_{m} s ( m ) ω ( n - m ) {e}^{- j ω im}$$

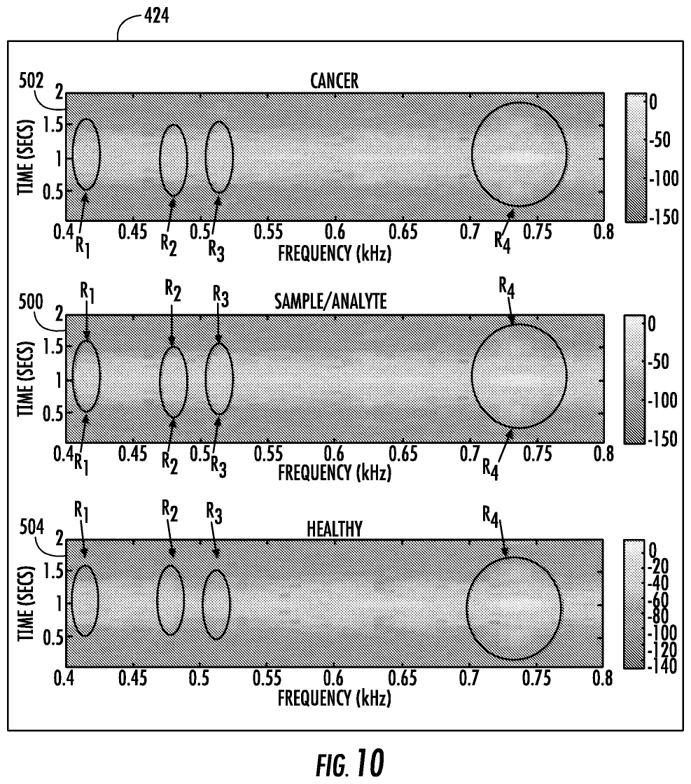

The time frequency representation by the spectrogram facilitates capture of the temporal-spectral behavior of time-domain signal used to approximate the data, such as SERS spectra. The conversion of the time domain response to a time frequency graphical representation in the form of a spectrogram using short time Fourier transform results in those components of the FIR time-domain signal that fit narrow peaks ringing longer whereas components that fit wider peaks ring” less in duration. FIG. 9 illustrates an example of the application of the windowed short time Fourier transform (with overlapping windows) to the time domain response for the cancerous sample shown in FIG. 6B.

As indicated by broken lines, in other implementations, block 309-311 may be replaced with alternative steps to convert the time domain response to other time-frequency representations. For example, in some implementations, in lieu of being converted to a spectrogram, the time-domain response may be converted to a Wigner-Ville time frequency representation which is then utilized as a basis for generating the color map.

As indicated by block 312, In the example illustrated, the time frequency representation may be transformed to red/green/blue channels representing an image of the spectrogram. In other implementations, other “color conversions may be applied to the spectrogram, such that the spectrogram is represented by other colors or in grayscale.

As indicated by block 316, processor 26, following instructions provided by computer vision analyzer 46, analyzes the color map and identifies values for predetermined optical characteristics or optical parameters (selected portions) of the time frequency graphical representation/color map through computer vision. In one implementation, computer vision analyzer 46 comprise a cascade of convolution on neural networks in a fully connected artificial neural network for identifying predetermined characteristics of the time frequency graphical representation. In other implementations, computer vision analyzer 46 may comprise instructions to direct processor 26 to utilize other computer vision techniques, such as SVM, Bayes classifier, etc. to identify predetermined characteristics of the time graphical representation, the color map.

In one implementation, the convolution of neural networks is trained on a graphical processing unit (GPU) with stochastic gradient descent with momentum update, dropout (to improve convergence and reduce the chance of network being stuck in minima), and in many-batch mode until a maximum number of epochs is reached. In such an implementation, healthy versus cancerous samples or fluid are discriminated by the computer vision (convoluted neural network) based upon differences in the temporal spreading (as circled in FIG. 10). Temporal spreading is a vertical height or vertical spreading. In the example illustrated, the cancerous sample has a greater degree of “temporal spreading” as compared to the healthy sample.

In one implementation, the spectrogram's and resulting color maps may be generated using a Hamming window (frame size) of 512 samples, with an overlap of 75%, over a duration of 8192 sample impulse responses, whereas the FFT-sized is kept at 512 frequency bins. In such an implementation, the window size, FFT size and overlap impact the dimensions of the color map. In one implementation, the image or color map has a dimension of 61×257 (height×width) with three channels (R/G/B). Images are provided as input to a receptive field of 61×257×3 of a first layer comprising of a 2-d CNN having filter size of 5×5 (height×width) with 30 filters. In such an implementation, the CNN layer comprises neurons that connect to small regions of the input or layer before it. Such regions are called filters. The number of filters represent the number of neurons in the convolution a layer that connect to the same region in the input and determines the number of channels (feature maps) in the output of the convolution a layer. The stride (traverse step in the height and width dimension) for each of the images is set to unitary. For each region, a dot product of the weights in the input is computed in a bias term is added. The filter moves along the input vertically and horizontally, repeating the same computations for each region, i.e., convoluting the input. The step size with which it moves is called a stride.

The number weights used for a filter is h×w×c, where H is a height, W is the width of the filter size and C is a number of channels in the input. For example, if the input is a color image, the number of channels is three corresponding to R/G/B. as a filter moves along the input, the filter uses the same set of weights and bias for the convolution, forming a future map. The CNN layer may have multiple feature maps, each with a different set of weights and a bias. The number of feature maps determined by the number of filters. The total number of parameters in a convolution a layer is ((h×w×c+1)×Number of Filters, where unity is for the bias.

The output from the neurons are passed through a nonlinearity which, in one implementation, may comprise a layer of rectified linear units (ReLU). The output from the ReLU layer is applied to a maximum pooling layer that down samples by a factor of two (using a stride parameter set 2). The height and width of the rectangular region (pool size) are set to 2. As a result, layer crates pooling regions of size [2, 2] returns a maximum of four elements in each region. Because the stride (step size for moving along the images vertically and horizontally) is also [2, 2], the pooling regions do not overlap in this layer.

In one implementation, a second 2-d CNN layer (height×width=5×5 with 20 filters) may be used to process the output from the max pooling layer with the output being delivered to a layer of ReLu and then a max pooling layer of height×width=2×2 that further down samples by a factor of 2. The output may then be applied to a fully connected feedforward neural network with two outputs to classify between cancer and healthy SERS data.

As indicated by arrow 317, processor 26, operating in a fluid classification building or supplementing mode (branch 302) pursuant to instructions provided by fluid identifier 48, stores the determined or identified values for the predetermined characteristics/parameters of the color map along with the associated (previously identified) substance or fluid to form a pre-trained convoluted neural network model 328, which may serve as or be part of the database/library 28 described above. In one implementation, the identified values for the predetermined characteristics are used to establish new library or database entries for the previously identified substance or fluid. In another implementation, the identified values for the predetermined characteristics are used to establish a larger statistical base for the value or range of values for each of the predetermined characteristics or parameters that are used to identify an unknown fluid as being of the same classification or type as the previously identified substance or fluid.

As shown by FIG. 5, branch 303 comprises the same blocks except that such actions are performed with respect to SERS spectra test data obtained from a fluid or sample to be classified. As indicated by block 404, processor 26 receives SERS spectra test data. After generating a color map based upon the SERS spectra test data, computer vision is utilized to extract values for selected portions of the color map for comparison to the model 328 generated pursuant to branch 302. The comparison is utilized to classify the SERS spectra test data and therefore classify the sample from which the test data was obtained.

As indicated by block 332, the classification of the unknown fluid (from which the SERS spectra test data was obtained) is presented using an indicator, such as indicator 24. As illustrated by FIG. 10, in one implementation, the basis for the classification is visibly presented to a user. FIG. 10 illustrates an example display 424, serving as indicator 24 described above. As indicated by block 334, when operating in one user selectable mode, processing unit 26, following instructions contained in fluid identifier 48, presents the color map for the sample/fluid being classified adjacent to or alongside at least one additional color map obtained from model 328 or library 28 such that the user may visibly ascertain the basis for the classification of the sample/fluid. As indicated by block 336, when operating in another user selectable mode, the color map for the sample/fluid being classified and the color map obtained from model 328 or library 28 which most closely corresponds to the color map of the sample being classified may be presented in and overlapping fashion, facilitating visible appreciation as to the similarities between the two color maps.

FIG. 10 illustrates an example of the side-by-side or adjacent positioning of color maps pursuant to block 334. FIG. 10 illustrates the display, on display 424, of an example color map 500 for a sample or fluid being classified, the color map 500 being generated pursuant to branch 303 of method 300. FIG. 10 illustrates the concurrent display, on display 424, of example color maps 502, 504 for classifications or predetermined fluid types generated pursuant to branch 302 of method 300. Color map 502 is an example color map generated pursuant to branch 302 from a sample predetermined to be cancerous. Color map 504 is an example color map generated pursuant to branch 302 from a sample predetermined to be “healthy”. In the example illustrated, the color map 500 is more similar to color map 502 than color map 504. In the example illustrated, processing unit 26 what output an indication that sample 500 has been determined to more likely than not be cancerous. The concurrent display of color map 500 along with color map 502 and/or 504 facilitates visual confirmation or understanding of the classification by the user. In some implementations, processing unit 26 may present the color map 500 with just the color map identified as being closest to color map 500, color map 502 in the example illustrated.

As further shown by FIG. 10, in the example illustrated, processing unit 26, following instructions contained in fluid identifier 48, may additionally visibly indicate those corresponding regions R of the different color maps that were compared to one another as part of the classification of the sample from which color map 500 was generated. In the example illustrated, display 424 identifies four distinct corresponding regions R1, R2, R3 and R4 on the color maps 500, 502 and 504 which were compared against one another. In the example illustrated, regions are identified by displayed ovals or circles annotating the color maps. In other implementations, the regions (predetermined characteristics of the color maps or signature characteristics) may be visibly indicated in other fashions. The visible indications facilitate a more focused review of the color maps by the person using system 20 and the color maps presented on display 424.

Although the present disclosure has been described with reference to example implementations, workers skilled in the art will recognize that changes may be made in form and detail without departing from the scope of the claimed subject matter. For example, although different example implementations may have been described as including features providing benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A method for fluid classification, the method comprising:
receiving sensed data for an unknown fluid;
modeling the sensed data in a frequency domain;
synthesizing a model of the sensed data from the frequency domain to a time domain response;
converting the time domain response to a time frequency graphical representation, wherein the time frequency graphical representation comprises a color map;
identifying predetermined characteristics of the time frequency graphical representation through computer vision; and
classifying the unknown fluid by comparing identified characteristics of the graphical representation to at least one corresponding signature characteristic of a predetermined fluid type.

2. The method of claim 1, wherein the sensed data comprises spectrographic data.

3. The method of claim 2, wherein the computer vision comprises applying a convoluted neural network to the graphical representation.

4. The method of claim 3, wherein converting the sensed data to a time frequency graphical representation comprises generating a spectrogram by:
windowing the time-domain synthesis; and
applying a Fourier transform to the windowed time-domain synthesis.

5. The method of claim 1 further comprising identifying the at least one corresponding signature characteristic of the predetermined fluid type by:
receiving second sensed data for the predetermined fluid type;
modeling the second sensed data in a frequency domain;
synthesizing a model of the second sensed data from the frequency domain to a time domain response;
converting the time domain response for the second sensed data to a second time-frequency graphical representation, wherein the second time-frequency graphical representation comprises a second color map;
identifying predetermined characteristics of the second time-frequency graphical representation through computer vision; and
store an association of the second identified characteristics of the second graphical representation to at least one signature characteristic of the predetermined fluid type.

6. The method of claim 5, wherein the sensed second data comprises second spectrographic data.

7. The method of claim 5, wherein the computer vision comprises applying a convoluted neural network to the second graphical representation.

8. The method of claim 1, wherein the computer vision comprises applying a convoluted neural network to the graphical representation.

9. The method of claim 1 further comprising concurrently displaying the color map with the identified predetermined characteristics being indicated and a second corresponding color map of the predetermined fluid type with the corresponding predetermined characteristics being indicated.

10. The method of claim 9, wherein the color map and the second corresponding color map are displayed adjacent to one another.

11. The method of claim 1, wherein the unknown fluid is classified as not being the predetermined fluid type based upon the comparing of the identified characteristics of the graphical representation to the at least one signature characteristic of the predetermined fluid type.

12. The method of claim 1, wherein the sensed data comprises spectrographic data.

13. A non-transitory computer-readable medium containing instructions to direct a processing unit to perform fluid classification by:
receiving sensed data for the fluid;
modeling the sensed data in a frequency domain;
synthesizing a model of the sensed data from the frequency domain to a time domain response;
converting the time domain response to a time frequency graphical representation, wherein the time frequency graphical representation comprises a color map;
identifying predetermined characteristics of the time frequency graphical representation through computer vision;
classifying the fluid by comparing identified characteristics of the graphical representation to at least one corresponding signature characteristic of a predetermined fluid type.

14. The non-transitory computer-readable medium of claim 13, wherein the sensed data comprises spectrographic data.

15. A database for fluid classification, the database comprising:
fluid classifications, each fluid classification comprising predetermined visual characteristics of the fluid classification corresponding to application of a convoluted neural network to a time-frequency representation of spectrographic data for the fluid classification.
